(19) United States
(12) Patent Application Publication
(10) Pub. No.: US 2008/0139598 A1
(43) Pub. Date: Jun. 12, 2008

(54) Amine-linked Multicyclic Compounds and Methods of Their Use

(76) Inventors: Joseph Barbosa, Lambertville, NJ (US); Yingzhi Bi, Plainsboro, NJ (US); Cynthia Anne Fink, Lebanon, NJ (US); Jian Cheng Wang, Addison, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 11/935,011

(22) Filed: Nov. 5, 2007

Related U.S. Application Data
(60) Provisional application No. 60/857,389, filed on Nov. 7, 2006.

(51) Int. Cl. A61K 31/506; C07D 239/24; A61P 25/00

(52) U.S. Cl. 514/275; 544/331

(57) ABSTRACT

This invention relates to multicyclic compounds, pharmaceutical compositions comprising them, and methods of their use. Particular compounds of the invention are of formula I:

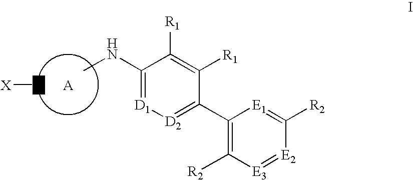

This application claims priority to U.S. provisional application No. 60/857,389, filed Nov. 7, 2006, the entirety of which is incorporated herein by reference.

### 1. FIELD OF THE INVENTION

This invention relates to multicyclic compounds, pharmaceutical compositions comprising them, and methods of their use.

### 2. BACKGROUND OF THE INVENTION

The amino acid L-proline reportedly plays a role in regulating synaptic transmission in the mammalian brain. See, e.g., Crump et al., Molecular and Cellular Neuroscience, 13: 25-29 (1999). For example, a synaptosomal bisynthetic pathway of L-proline from ornithine has been reported, and high affinity Na⁺-dependent synaptosomal uptake of L-proline has been observed. Yoneda et al., Brain Res., 239: 479-488 (1982); Balcar et al., Brain Res., 102: 143-151 (1976).

In general, neurotransmitter systems typically have mechanisms that inactivate signaling, many of which work through the action of a Na⁺-dependent transporter. In this case, a Na⁺-dependent transporter for proline has been described, and the molecular entity cloned (SLC6A7 in humans). See, e.g., U.S. Pat. Nos. 5,580,775 and 5,759,788. But the transporter's specific role remains unknown. For example, the human Na⁺-dependent proline transporter is generally localized to synaptic terminals, which is consistent with a role in neurotransmitter signaling. But no high-affinity receptor has been found for proline, suggesting that it is a neuromodulator rather than a neurotransmitter. Shafqat S., et al., Molecular Pharmacology 48:219-229 (1995).

The fact that the Na⁺-dependent proline transporter is expressed in the dorsal root ganglion has led some to suggest that it may be involved in nociception, and that compounds which inhibit the transporter may be used to treat pain. See, e.g., U.S. Patent Application No. 20030152970A1. But this suggestion is not supported by experimental data.

### 3. SUMMARY OF THE INVENTION

This invention encompasses multicyclic compounds, pharmaceutical compositions comprising them, and methods of their use. One embodiment of the invention encompasses a compound of formula I:

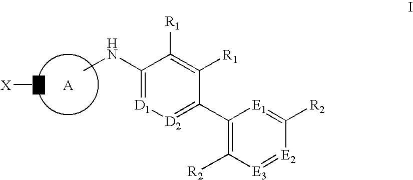

and pharmaceutically acceptable salts and solvates thereof, wherein: A is an optionally substituted non-aromatic heterocycle; each of D₁ and D₂ is independently N or CR₁; each of E₁, E₂ and E₃ is independently N or CR₂; X is optionally substituted heteroaryl; each R₁ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; each R₂ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; each R_{A} is independently hydrogen or optionally substituted alkyl, aryl, arylalkyl, alkylaryl, heterocycle, heterocycle-alkyl, or alkyl-heterocycle; and each R_{B} is independently hydrogen or optionally substituted alkyl, aryl, arylalkyl, alkylaryl, heterocycle, heterocycle-alkyl, or alkyl-heterocycle.

Preferred compounds inhibit the proline transporter, and particular compounds do so without substantially affecting the dopamine or glycine transporters.

Another embodiment of the invention encompasses pharmaceutical compositions of the various compounds described herein.

Another embodiment encompasses methods of improving cognitive performance, and of treating, managing and/or preventing various diseases and disorders, using compounds of the invention.

### 4. DETAILED DESCRIPTION OF THE INVENTION

This invention is based, in part, on the discovery that the proline transporter encoded by the human gene at map location 5q31-q32 (SLC6A7 gene; GENBANK accession no. NM_{—}014228) can be a potent modulator of mental performance in mammals. In particular, it has been found that genetically engineered mice that do not express a functional product of the murine ortholog of the SLC6A7 gene display significantly increased cognitive function, attention span, learning, and memory relative to control animals. See U.S. patent application Ser. Nos. 11/433,057 and 11/433,626, both filed May 12, 2006.

In view of this discovery, the protein product associated with the SLC6A7 coding region was used to discover compounds that may improve cognitive performance and may be useful in the treatment, prevention and/or management of diseases and disorders such as Alzheimer's disease, autism, cognitive disorders, dementia, learning disorders, and short- and long-term memory loss.

### 4.1. Definitions

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “alkoxy” means an —O-alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃.

Unless otherwise indicated, the term “aryl” means an aromatic ring or an aromatic or partially aromatic ring system composed of carbon and hydrogen atoms. An aryl moiety may comprise multiple rings bound or fused together. Examples of aryl moieties include anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the term “DTIC₅₀” means an IC₅₀ against human recombinant dopamine transporter as determined using the assay described in the Examples, below.

Unless otherwise indicated, the term “GTIC₅₀” means an IC₅₀ for human recombinant glycine transporter as determined using the assay described in the Examples, below.

Unless otherwise indicated, the terms “halogen” and “halo” encompass fluorine, chlorine, bromine, and iodine.

Unless otherwise indicated, the term “heteroalkyl” refers to an alkyl moiety (e.g., linear, branched or cyclic) in which at least one of its carbon atoms has been replaced with a heteroatom (e.g., N, O or S).

Unless otherwise indicated, the term “heteroaryl” means an aryl moiety wherein at least one of its carbon atoms has been replaced with a heteroatom (e.g., N, O or S). Examples include acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moiety.

Unless otherwise indicated, the term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkyl-alkyl” refers to a heterocycloalkyl moiety bound to an alkyl moiety.

Unless otherwise indicated, the terms “manage,” “managing” and “management” encompass preventing the recurrence of the specified disease or disorder, or of one or more of its symptoms, in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences (18th ed., Mack Publishing, Easton Pa.: 1990) and Remington: The Science and Practice of Pharmacy (19th ed., Mack Publishing, Easton Pa.: 1995).

Unless otherwise indicated, the term “potent proline transporter inhibitor” means a compound that has a PTIC₅₀ of less than about 200 nM.

Unless otherwise indicated, the terms “prevent,” “preventing” and “prevention” contemplate an action that occurs before a patient begins to suffer from the specified disease or disorder, which inhibits or reduces the severity of the disease or disorder, or of one or more of its symptoms. The terms encompass prophylaxis.

Unless otherwise indicated, a “prophylactically effective amount” of a compound is an amount sufficient to prevent a disease or condition, or one or more symptoms associated with the disease or condition, or to prevent its recurrence. A prophylactically effective amount of a compound is an amount of therapeutic agent, alone or in combination with other agents, which provides a prophylactic benefit in the prevention of the disease or condition. The term “prophylactically effective amount” can encompass an amount that improves overall prophylaxis or enhances the prophylactic efficacy of another prophylactic agent.

Unless otherwise indicated, the term “PTIC₅₀” means an IC₅₀ for human recombinant Na⁺-dependent proline transporter as determined using the assay described in the Examples, below.

Unless otherwise indicated, the term “potent proline transporter inhibitor” means a compound that has a PTIC₅₀ of less than about 200 nM.

Unless otherwise indicated, the term “stereomerically enriched composition of” a compound refers to a mixture of the named compound and its stereoisomer(s) that contains more of the named compound than its stereoisomer(s). For example, a stereoisomerically enriched composition of (S)-butan-2-ol encompasses mixtures of (S)-butan-2-ol and (R)-butan-2-ol in ratios of, e.g., about 60/40, 70/30, 80/20, 90/10, 95/5, and 98/2.

Unless otherwise indicated, the term “stereomerically pure” means a composition that comprises one stereoisomer of a compound and is substantially free of other stereoisomers of that compound. For example, a stereomerically pure composition of a compound having one stereocenter will be substantially free of the opposite stereoisomer of the compound. A stereomerically pure composition of a compound having two stereocenters will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound.

Unless otherwise indicated, the term “substituted,” when used to describe a chemical structure or moiety, refers to a derivative of that structure or moiety wherein one or more of its hydrogen atoms is substituted with a chemical moiety or functional group such as, but not limited to, alcohol, aldehyde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH₂), amine (primary, secondary and tertiary such as alkylamino, arylamino, arylalkylamino), aroyl, aryl, aryloxy, azo, carbamoyl (—NHC(O)O-alkyl- or —OC(O)NH-alkyl), carbamyl (e.g., CONH₂, CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-).

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound is an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or one or more of its symptoms, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit. Structures that represent compounds with one or more chiral centers, but which do not indicate stereochemistry (e.g., with bolded or dashed lines), encompasses pure stereoisomers and mixtures (e.g., racemic mixtures) thereof. Similarly, names of compounds having one or more chiral centers that do not specify the stereochemistry of those centers encompass pure stereoisomers and mixtures thereof.

### 4.2. Compounds of the Invention

This invention encompasses compounds of formula I:

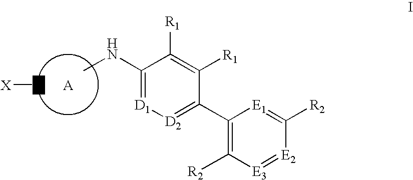

and pharmaceutically acceptable salts and solvates thereof, wherein: A is an optionally substituted non-aromatic heterocycle; each of D₁ and D₂ is independently N or CR₁; each of E₁, E₂ and E₃ is independently N or CR₂; X is optionally substituted heteroaryl; each R₁ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; each R₂ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; each R_{A} is independently hydrogen or optionally substituted alkyl, aryl, arylalkyl, alkylaryl, heterocycle, heterocycle-alkyl, or alkyl-heterocycle; and each R_{B} is independently hydrogen or optionally substituted alkyl, aryl, arylalkyl, alkylaryl, heterocycle, heterocycle-alkyl, or alkyl-heterocycle.

In one embodiment, A is monocyclic. In another, A is bicyclic. In another, A is unsubstituted. In another, A is optionally substituted pyrrolidine, piperidine, hexahydropyrimidine, 1,2,3,6-tetrahydropyridine, octahydrocyclopenta[c]pyrrole, or octahydropyrrolo[3,4-c]pyrrole.

In one embodiment, one of D₁ and D₂ is N. In another, both D₁ and D₂ are N. In another, both D₁ and D₂ are CR₁.

In one embodiment, one of E₁, E₂ and E₃ is N. In another, two of E₁, E₂ and E₃ are N. In another, all of E₁, E₂ and E₃ are N. In another, all of E₁, E₂ and E₃ are independently CR₂.

In one embodiment, R₁ is hydrogen, halogen, or optionally substituted alkyl. In another, R₁ is OR_{A} and R_{A} is, for example, hydrogen or optionally substituted alkyl.

In one embodiment, R₂ is hydrogen, halogen, or optionally substituted alkyl. In another, R₂ is OR_{A} and R_{A} is, for example, hydrogen or optionally substituted alkyl.

In one embodiment, X is an optionally substituted 5-, 6-, 9- or 10-membered heteroaryl. In another, X is optionally substituted 5- or 6-membered heteroaryl. In another, X is of the formula:

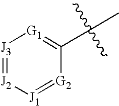

wherein: each of G₁ and G₂ are independently N or CR₃; each of J₁, J₂ and J₃ are independently N or CR₄; each R₃ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; and each R₄ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; provided that at least one of J₁, J₂ and J₃ is CR₄.

In a particular embodiment, one of G₁ and G₂ is N. In another, both G₁ and G₂ are N. In another, both G₁ and G₂ are CR₃. In another, one of J₁, J₂ and J₃ is N. In another, two of J₁, J₂ and J₃ are N. In another, all of J₁, J₂ and J₃ are independently CR₄.

In one embodiment, R₃ is hydrogen, halogen, or optionally substituted alkyl. In another, R₃ is OR_{A} and R_{A} is, for example, hydrogen or optionally substituted alkyl.

In one embodiment, R₄ is hydrogen, halogen, or optionally substituted alkyl. In another, R₄ is OR_{A} and R_{A} is, for example, hydrogen or optionally substituted alkyl.

One embodiment of the invention encompasses compounds of formula I(A):

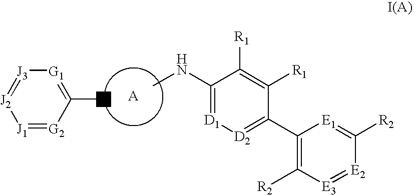

and pharmaceutically acceptable salts and solvates thereof.

Another encompasses compounds of formula I(B):

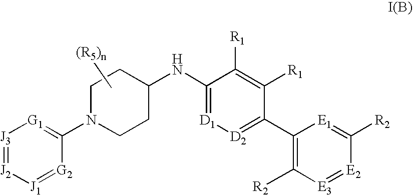

and pharmaceutically acceptable salts and solvates thereof, wherein: each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and n is 0-5.

Another encompasses compounds of formula I(C):

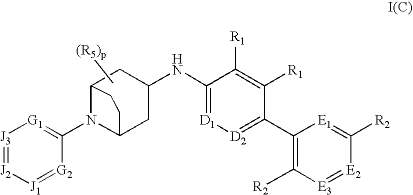

and pharmaceutically acceptable salts and solvates thereof, wherein: each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and p is 0-7.

Another encompasses compounds of formula I(D):

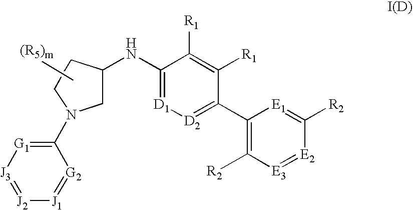

and pharmaceutically acceptable salts and solvates thereof, wherein: each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A}; each R_{5A} is independently hydrogen or optionally substituted alkyl, aryl, arylalkyl, alkylaryl, heterocycle, heterocycle-alkyl, or alkyl-heterocycle; each R_{5B} is independently hydrogen or optionally substituted alkyl, aryl, arylalkyl, alkylaryl, heterocycle, heterocycle-alkyl, or alkyl-heterocycle; and m is 0-4.

Compounds of the invention may contain one or more stereocenters, and can exist as mixtures of enantiomers or diastereomers. This invention encompasses stereomerically pure forms of such compounds and stereomerically enriched compositions thereof. Stereoisomers may be asymmetrically synthesized or resolved using standard techniques such as chiral columns or chiral resolving agents. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, NY, 1962); and Wilen, S. H., Tables of Resolving Agents and Optical Resolutions, p. 268 (E. L. Eliel, Ed., Univ. of Notre Dame Press, Notre Dame, IN, 1972).

Examples of compounds encompassed by the invention include:
N-(3′-chloro-3-methylbiphenyl-4-yl)-1-(pyrimidin-2-yl)piperidin-4-amine;
N-(4′-chloro-2′-fluorobiphenyl-4-yl)-8-(pyrimidin-2-yl)-8-azabicyclo[3.2.1]octan-3-amine;
N-(2′,4′-difluorobiphenyl-4-yl)-1-(pyrimidin-2-yl)piperidin-4-amine;
(3′-chloro-3-nitro-biphenyl-4-yl)-(1-pyrimidin-2-yl-piperidin-4-yl)-amine;
3′-chloro-N⁴-(1-(pyrimidin-2-yl)piperidin-4-yl)biphenyl-3,4-diamine;
N⁴-(1-(pyrimidin-2-yl)piperidin-4-yl)biphenyl-3,4-diamine;
(3′-chloro-3-cyano-biphenyl-4-yl)-(1-pyrimidin-2-yl-piperidin-4-yl)-amine;
(3′-chloro-2-methoxy-biphenyl-4-yl)-(1-pyrimidin-2-yl-piperidin-4-yl)-amine;
(3′-chloro-3-chloro-biphenyl-4-yl)-(1-pyrimidin-2-yl-piperidin-4-yl)-amine;
(3′-chloro-2-chloro-biphenyl-4-yl)-(1-pyrimidin-2-yl-piperidin-4-yl)-amine;
(2,3′-dichloro-3-methyl-biphenyl-4-yl)-(1-pyrimidin-2-yl-piperidin-4-yl)-amine;
(3′-chloro-3-trifluoromethyl-biphenyl-4-yl)-(1-pyrimidin-2-yl-piperidin-4-yl)-amine;
(3′-chloro-2-trifluoromethyl-biphenyl-4-yl)-(1-pyrimidin-2-yl-piperidin-4-yl)-amine;
(3′-chloro-3-methyl-biphenyl-4-yl)-(1-pyrimidin-2-yl-piperidin-4-yl)-amine;
(3′-chloro-2-methyl-biphenyl-4-yl)-(1-pyrimidin-2-yl-piperidin-4-yl)-amine;
(3′-chloro-3-fluoro-biphenyl-4-yl)-(1-pyrimidin-2-yl-piperidin-4-yl)-amine;
(3′-chloro-2-fluoro-biphenyl-4-yl)-(1-pyrimidin-2-yl-piperidin-4-yl)-amine;
(3-cyano-2′,4′-difluoro-biphenyl-4-yl)-(1-pyrimidin-2-yl-piperidin-4-yl)-amine;
(2′,4′-difluoro-2-methoxy-biphenyl-4-yl)-(1-pyrimidin-2-yl-piperidin-4-yl)-amine;
(3-chloro-2′4′-difluoro-biphenyl-4-yl)-(1-pyrimidin-2-yl-piperidin-4-yl)-amine;
(2-chloro-2′,4′-difluoro-biphenyl-4-yl)-(1-pyrimidin-2-yl-piperidin-4-yl)-amine;
(2-chloro-2′,4′-difluoro-3-methyl-biphenyl-4-yl)-(1-pyrimidin-2-yl-piperidin-4-yl)-amine;
(2′,4′-difluoro-3-trifluoromethyl-biphenyl-4-yl)-(1-pyrimidin-2-yl-piperidin-4-yl)-amine;
(2′,4′-difluoro-2-trifluoromethyl-biphenyl-4-yl)-(1-pyrimidin-2-yl-piperidin-4-yl)-amine;
(2′,4′-difluoro-3-methyl-biphenyl-4-yl)-(1-pyrimidin-2-yl-piperidin-4-yl)-amine;
(2′,4′-difluoro-2-methyl-biphenyl-4-yl)-(1-pyrimidin-2-yl-piperidin-4-yl)-amine;
(1-pyrimidin-2-yl-piperidin-4-yl)-(3,2′,4′-trifluoro-biphenyl-4-yl)-amine;
(1-pyrimidin-2-yl-piperidin-4-yl)-(2,2′,4′-trifluoro-biphenyl-4-yl)-amine;
(5-chloro-2′-fluoro-3-methyl-biphenyl-4-yl)-(1-pyrimidin-2-yl-piperidin-4-yl)-amine;
(5′-chloro-3,2′-difluoro-biphenyl-4-yl)-(1-pyrimidin-2-yl-piperidin-4-yl)-amine;
(5′-chloro-2,2′-difluoro-biphenyl-4-yl)-(1-pyrimidin-2-yl-piperidin-4-yl)-amine;
(4′-chloro-3-methyl-biphenyl-4-yl)-(1-pyrimidin-2-yl-piperidin-4-yl)-amine;
(4′-chloro-2-methyl-biphenyl-4-yl)-(1-pyrimidin-2-yl-piperidin-4-yl)-amine;
(4′-chloro-3-fluoro-biphenyl-4-yl)-(1-pyrimidin-2-yl-piperidin-4-yl)-amine; and
(4′-chloro-2-fluoro-biphenyl-4-yl)-(1-pyrimidin-2-yl-piperidin-4-yl)-amine.

Preferred compounds of the invention are potent proline transporter inhibitors. Particular potent proline transporter inhibitors have a PTIC₅₀ of less than about 150, 125, 100, 75, 50 or 25 nM.

Some compounds inhibit the murine Na⁺-dependent proline transporter, as determined by the method described in the Examples below, with an IC₅₀ of less than about 150, 125, 100, 75, 50 or 25 nM.

Some compounds do not significantly inhibit the dopamine transporter. For example, some potent proline transporter inhibitors inhibit the dopamine transporter with an IC₅₀ of greater than about 0.5, 1, 2.5, 5, or 10 μM as determined using the assay described in the Examples below.

Some compounds do not significantly inhibit the glycine transporter. For example, some potent proline transporter inhibitors inhibit the glycine transporter with an IC₅₀ of greater than about 0.5, 1, 2.5, 5, or 10 μM as determined using the assay described in the Examples below.

### 4.3. Preparation of Compounds

Compounds of the invention may be obtained or prepared using synthetic methods known in the art, as well as those described herein. For example, compounds of formula I can be prepared by the general method shown below in Scheme 1:

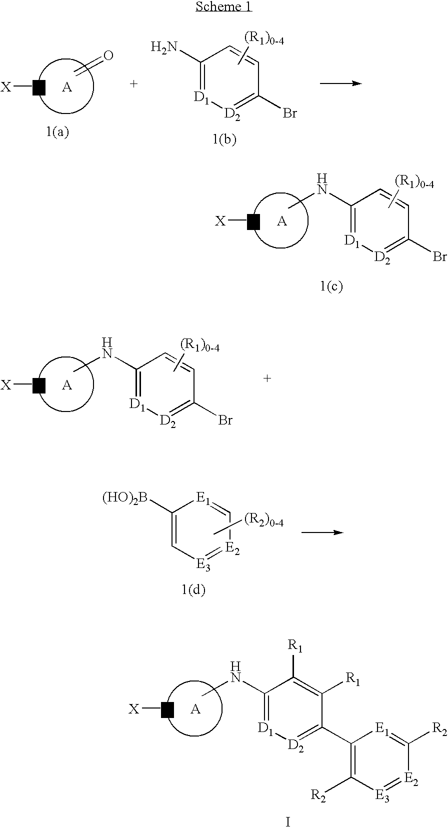

In this approach, compound 1(a), which is readily prepared by known methods or is commercially available, is coupled with compound 1(b), which is readily prepared by known methods or is commercially available, under suitable conditions (e.g., BH₃ Py in an appropriate solvent.) The resulting compound 1(c) is then coupled with compound 1(d), which is readily prepared by known methods or is commercially available, under Suzuki coupling conditions to provide a compound of formula I.

Some specific reaction conditions that can be used in the various synthetic schemes shown above are provided in the Examples, below.

### 4.4. Methods of Treatment

One embodiment of this invention encompasses a method of inhibiting a proline transporter, which comprises contacting a proline transporter (in vitro or in vivo) with a sufficient amount of a compound of the invention. Preferred proline transporters are encoded by the human gene SLC6A7, the murine ortholog thereof, or a nucleic acid molecule that encodes a proline transporter and that hybridizes under standard conditions to the full length of either.

Another embodiment encompasses a method of improving the cognitive performance of a human patient, which comprises administering to the patient an effective amount of a compound of the invention. Examples of improved cognitive performance include enhanced learning (e.g., learning more quickly), improved comprehension, improved reasoning, and improved short- and/or long-term memory.

Another embodiment encompasses a method of treating, managing or preventing a cognitive disorder (e.g., difficulty in thinking, reasoning, or problem solving), memory loss (short- and long-term), or a learning disorder (e.g., dyslexia, dyscalculia, dysgraphia, dysphasia, dysnomia), which comprises administering to the patient an effective amount of a compound of the invention.

Another embodiment encompasses a method of treating, managing or preventing a disease or disorder, or a cognitive impairment associated therewith, in a human patient, which comprises administering to the patient a therapeutically or prophylactically effective amount of a compound of the invention. Examples of diseases and disorders include age-associated memory impairment, Alzheimer's disease, Attention-Deficit/Hyperactivity Disorder (ADD/ADHD), autism, Down syndrome, Fragile X syndrome, Huntington's disease, Parkinson's disease, and schizophrenia. Additional disorders include adverse sequelae of brain damage caused by, for example, oxygen starvation, traumatic injury, heart attack or stroke.

The invention also encompasses methods of treating, preventing and managing dementia, including dementia associated with metabolic-toxic, structural and/or infectious causes.

Metabolic-toxic causes of dementia include: anoxia; B₁₂ deficiency; chronic drug, alcohol or nutritional abuse; folic acid deficiency; hypercalcemia associated with hyperparathyroidism; hypoglycemia; hypothyroidism; organ system failure (e.g., hepatic, respiratory, or uremic encephalopathy); and pellagra.

Structural causes of dementia include: amyotrophic lateral sclerosis; brain trauma (e.g., chronic subdural hematoma, dementia pugilistica); brain tumors; cerebellar degeneration; communicating hydrocephalus; irradiation to frontal lobes; multiple sclerosis; normal-pressure hydrocephalus; Pick's disease; progressive multifocal leukoencephalopathy; progressive supranuclear palsy; surgery; vascular disease (e.g., multi-infarct dementia); and Wilson's disease.

Infectious causes of dementia include: bacterial endocarditis; Creutzfeldt-Jakob disease; Gerstmann-Sträussler-Scheinker disease; HIV-related disorders; neurosyphilis; tuberculous and fungal meningitis; and viral encephalitis.

### 4.5. Pharmaceutical Compositions

This invention encompasses pharmaceutical compositions and dosage forms comprising compounds of the invention as their active ingredients. Pharmaceutical compositions and dosage forms of this invention may optionally contain one or more pharmaceutically acceptable carriers or excipients. Certain pharmaceutical compositions are single unit dosage forms suitable for oral, topical, mucosal (e.g., nasal, pulmonary, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration may require enteric coatings to protect the active ingredient from degradation within the gastrointestinal tract. In another example, the active ingredient may be administered in a liposomal formulation to shield it from degradative enzymes, facilitate transport in circulatory system, and/or effect delivery across cell membranes to intracellular sites.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

### 5. EXAMPLES

### 5.1. Preparation of N-(3′-chloro-3-methylbiphenyl-4-yl)-1-(pyrimidin-2-yl)piperidin-4-amine

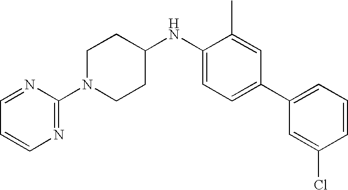

The title compound was prepared stepwise, as described below.

A. 1-Pyrimidin-2-yl-piperidin-4-one: A mixture of 4-piperidone monohydrate hydrochloride (4.84 g, 31.5 mmol), 2-chloropyrimidine (3.44 g, 30 mmol) and TEA (10.04 ml, 72 mmol) in EtOH (150 ml) was heated at reflux for overnight. The mixture was concentrated to almost dry and diluted with EtOAc (400 ml). The EtOAc layer was washed with water (2×50 ml) and brine (2×50 ml). The aq layer was back extracted with EtOAc (4×100 ml). The combined EtOAc was dried (Na₂SO₄) and the solvent was removed. The residue was subjected to ISCO (120 g column, hexane 5 min., 0-80% EtOAc in hexane over 70 min., then EtOAc for 15 min) to give the titled compound (3.5 g, 66%). HPLC: column, Luna Phenyl-Hexyl 5 μm 4.6×50 mm, 10-90% solvent B (acetonitrile) in solvent A (10 mM ammonium acetate aq.) over 3 min., flow rate 3 ml/min, retention time, 0.97 and 1.08 min.; MS (MH⁺: 178).

¹H NMR (300 MHz, chloroform-d), δ ppm 2.52 (t, J=6.29 Hz, 4H), 4.15 (t, J=6.20 Hz, 4H), 6.60 (t, J=4.67 Hz, 1H), 8.38 (d, J=4.77 Hz, 2H).

B. (4-Bromo-2-methyl-phenyl)-(1-pyrimidin-2-yl-piperidin-4-yl)-amine: To the mixture of 4-bromo-2-methylaniline (285 mg, 1.5 mmol) and 1-pyrimidin-2-yl-piperidin-4-one (266 mg, 1.5 mmol) in MeOH/AcOH (10:1, 5 ml) was added BH₃.Py in THF (8M, 188 μl). The mixture was stirred at rt for 2 h. The reaction mixture was concentrated and to the residue was added HCl (10%, 10 ml). The resulting mixture was stirred at room temperature for 30 min., then with cooling, the mixture was adjusted to alkaline with solid Na₂CO₃ and water. The aqueous layer was extracted with EtOAc (5×30 ml). The EtOAc was dried (Na₂SO₄) and concentrated. The residue was subjected to ISCO to give the titled compound as a white solid (140 mg). HPLC: column, Luna Phenyl-Hexyl 5 μm 4.6×50 mm, 10-90% solvent B (acetonitrile) in solvent A (10 mM ammonium acetate aq) over 3 min., flow rate 3 ml/min, retention time, 2.71 min.; MS (MH⁺: 347 and 349).

¹H NMR (400 MHz, chloroform-d), δ ppm 1.46 (ddd, J=24.30, 10.86, 4.04 Hz, 2H), 2.10 (s, 3H), 2.17 (dd, J=13.14, 2.78 Hz, 2H), 3.20 (ddd, J=14.00, 11.87, 2.78 Hz, 1H), 3.40 (br. s., 1H), 3.60 (br. s., 1H), 4.68 (ddd, J=13.52, 3.41, 3.28 Hz, 2H), 6.50 (t, J=4.67 Hz, 1H), 6.57 (d, J=8.59 Hz, 1H), 7.19 (s, 1H), 7.23 (dd, J=8.59, 2.27 Hz, 1H), 8.33 (d, J=4.80 Hz, 2H).

C. (3′-Chloro-3-methyl-biphenyl-4-yl)-(1-pyrimidin-2-yl-piperidin-4-yl)-amine:

To a solution of (4-bromo-2-methyl-phenyl)-(1-pyrimidin-2-yl-piperidin-4-yl)-amine (100.0 mg, 0.288 mmol) in MeCN (4 ml) was added 3-chlorophenylboronic acid (43.8 mg, 0.346 mmol), K₂CO₃ (79.5 mg, 0.576 mmol), PdCl₂(PPh₃)₂ (10.5 mg, 0.015 mmol) and water (1 ml). This mixture was microwaved for 10 min at 140° C. The reaction mixture was diluted with 15 ml of EtOAc, washed with water and brine, and then dried over MgSO₄. It was concentrated and purified by preparative HPLC to obtain 86.0 mg (79%) of the product. HPLC: column, Shim-Pack VP ODS 4.6×50 mm, 10-90% solvent B (MeOH+0.1% TFA) in solvent A (Water+0.1% TFA) over 2 min., flow rate 3.5 ml/min, stop time 3 min, retention time, 2.49 min.; MS (MH⁺: 379).

¹HNMR (400 MHz, chloroform-d), δ ppm 1.49 (ddd, J=23.50, 11.14, 3.91 Hz, 2H), 2.18 (s, 4H), 2.22 (d, J=3.13 Hz, 1H), 3.21 (ddd, J=13.58, 11.23, 2.74 Hz, 2H), 3.60-3.74 (m, 1H), 4.68 (dt, J=13.68, 3.32 Hz, 2H), 6.49 (t, J=4.69 Hz, 1H), 6.75 (d, J=8.60 Hz, 1H), 7.21 (dt, J=8.89, 1.03 Hz, 1H), 7.30 (t, J=7.91 Hz, 1H), 7.31 (d, J=1.76 Hz, 1H), 7.36 (dd, J=8.40, 2.34 Hz, 1H), 7.42 (dd, J=7.82, 1.37 Hz, 1H), 7.53 (t, J=1.95 Hz, 1H), 8.32 (d, J=4.69 Hz, 2H).

### 5.2. Preparation of N-(4′-chloro-2′-fluorobiphenyl-4-yl)-8-(pyrimidin-2-yl)-8-azabicyclo[3.2.1]octan-3-amine

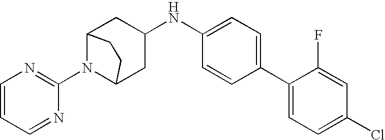

The title compound was prepared stepwise, as described below.

A. 8-Pyrimidin-2-yl-8-aza-bicyclo[3.2.1]octan-3-one (3): A mixture of 8-aza-bicyclo[3.2.1]octan-3-one hydrochloride (1) (5 g, 31 mmol), 2-chloropyrimidine (2) (4.3 g, 37 mmol), and sodium bicarbonate (7.8 g, 93 mmol) were stirred at reflux in 2-propanol (200 ml) for 48 h, filtered, evaporated and chromatographed (silica gel, 25% (v/v) EtOAc/hexane) to afford 4 grams.

B. (4-Bromo-phenyl)-(8-pyrimidin-2-yl-8-aza-bicyclo[3.2.1]oct-3-yl)-amine (6): Toluene and p-toluenesulfonic acid were added to 8-pyrimidin-2-yl-8-aza-bicyclo[3.2.1]octan-3-one (3) and 4-bromoaniline. This mixture was refluxed until the requisite volume of water was collected in a Dean—Stark trap, then evaporated and dissolved in ethanol. To this crude solution were added 2 equivalents of acetic acid and 1 equivalent of sodium cyanoborohydride and allowed to stir until reduction was complete. MS: M+H=359, 361.

C. (4′-Chloro-2′-fluoro-biphenyl-4-yl)-(8-pyrimidin-2-yl-8-aza-bicyclo[3.2.1]oct-3-yl)-amine: To a solution of (4-bromo-phenyl)-(8-pyrimidin-2-yl-8-aza-bicyclo[3.2.1]oct-3-yl)-amine (0.1 g, 0.3 mmol), 4-chloro-2-fluoro-phenylboronic acid (0.7 mg, 0.4 mmol), and potassium phosphate tribasic (1.0 g, 4.5 mmol) in a 3:1 volume solution of 1,2-dimethoxyethane and water was added [1,1′-bis(diphenyl phosphino)ferrocene]dichloropalladium, complex with dichloromethane (8 mg, 0.01 mmol). The mixture was heated to 80° C., cooled, poured into dichloromethane and washed with 1M aqueous sodium hydroxide. Product was purified by column chromatography (silica gel, 0 to 50% (v/v) EtOAc/hexane). MS: M+H=409.

¹H NMR (CDCl₃): δ ppm, d, j=13.9 Hz, 2H, 2.17 ppm, m, 4H, 2.38 ppm, m, 2H; 3.72 ppm, t, j=6.1 Hz, 1H, 4.29 ppm, br s, 1H, 4.80 ppm, s, 2H, 6.53 ppm, t, j=4.8 Hz, 1H; 6.62 ppm, d, j=6.8 Hz, 2H, 7.06 ppm, dd, j=8.8, 8.6 Hz, 1H, 7.19 ppm, m, 1H, 7.40 ppm, d, j=8.6 Hz, 3H, 8.37 ppm, d, j=4.8 Hz, 2H.

### 5.3. Preparation of N-(2′,4′-difluorobiphenyl-4-yl)-1-(pyrimidin-2-yl)piperidin-4-amine

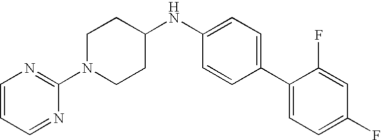

The title compound was prepared stepwise, as described below.

A. 1-Pyrimidin-2-yl-piperidin-4-one: 2-Propanol (125 ml) was added to a mixture of piperidine-4,4-diol hydrochloride (9.9 g, 64.6 mmol), 2-chloropyrimidine (9.4 g, 81.8 mmol) and sodium bicarbonate (21.8 g, 259.3 mmol). The rapidly stirred suspension was heated to reflux for 17 h, cooled, and filtered through celite, and evaporated to provide 13.2 g of clear yellow oil which was used without further purification. MS: M+H=178.

B. (4-Bromo-phenyl)-(1-pyrimidin-2-yl-piperidin-4-yl)-amine: To a solution of 1-pyrimidin-2-yl-piperidin-4-one (13.2 g, 74.5 mmol), 4-bromoaniline (12.8 g, 74.5 mmol), and acetic acid (8.5 ml, 150.0 mmol) in methanol (250 ml) was added powdered 4 Å molecular sieves (6.8 g). This suspension was rapidly stirred for 0.5 h then sodium cyanoborohydride (4.9 g, 74.5 mmol) was added and the mixture allowed to stir at ambient temperature, under N₂ blanket, for 17 h. The reaction mixture was filtered through celite. The filtrate, acidified to pH ca. 2 with 6M HCl (aq.) was then basified with 5% (w/v) NaHCO₃ (aq.) and extracted with dichloromethane. The organic extracts were dried (MgSO₄), filtered, evaporated, chromatographed (silica gel, elution solvent: 100% EtOAc) and crystallized (EtOAc/heptane) to afford 10.4 g (42%) of tan fine crystalline powder. MS: M+H=333.

C. (2′4′-Difluoro-biphenyl-4-yl)-(1-pyrimidin-2-yl-piperidin-4-yl)-amine: To a solution of (4-bromo-phenyl)-(1-pyrimidin-2-yl-piperidin-4-yl)-amine (2) (1.7 g, 5.1 mmol), and 2,4-difluorophenylboronic acid (1.0 g, 6.1 mmol) in 1,2-dimethoxyethane (50 ml) was added a solution of potassium phosphate tribasic (2.2 g, 10.1 mmol) in water (3 ml). The resultant solution was then taken through 10 evacuation/N₂ blanketing cycles. [1,1′-Bis-(diphenyl phosphino)ferrocene]-dichloropalladium(II) complex with dichloromethane (0.4 g, 0.5 mmol) was then added to the reaction flask and the system taken though another 10 evacuation/N₂ blanketing cycles. The rapidly stirred reaction was heated to reflux, under N₂ blanket, for 4 h then cooled, diluted with water and extracted with EtOAc. Extracts were washed with saturated NaCl (aq.), dried (MgSO₄), filtered, evaporated, flash chromatographed (silica gel, elution solvent: 15% (v/v) EtOAc/heptane), and crystallized (heptane) to yield 1.7 g (94%) of fibrous white crystals, m.p. 162-163° C. MS: M+H=367;

¹H NMR (d₆-DMSO): δ ppm, m, 2H, 1.99 ppm, d, j=10.1 Hz, 2H, 3.16 ppm, t, j=11.1 Hz, 2H, 3.59 ppm, m, 1H, 4.56 ppm, d, j=13.4 Hz, 2H, 5.77 ppm, d, j=8.3 Hz, 1H; 6.59 ppm, t, j=4.8 Hz, 1H, 6.71 ppm, d, j=8.8 Hz, 2H, 7.11 ppm, t, j=2.0 Hz, 1H, 7.26 ppm, m, 3H, 7.47 ppm, m, 1H, 8.36 ppm, d, j=4.8 Hz, 2H. Elemental analysis C₂₁H₂₀N₄F₂ C,H,N req'd: 68.82, 5.51, 15.27; fnd: 68.62, 5.36, 15.17.

### 5.4. Preparation of (3′-Chloro-3-nitro-biphenyl-4-yl)-(1-pyrimidin-2-yl-piperidin-4-yl)-amine

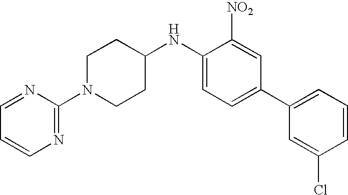

The title compound was obtained in the stepwise synthesis described below.

A. (1-Pyrimidin-2-yl-piperidin-4-yl)-carboxylic acid tert-butyl ester: To 5 g (25.0 mmol) of 4-Boc-aminopiperidine dissolved in 90 ml of EtOH was added 3.6 g (25.0 mmol) of 2-chloropyrimidine, and 3.45 ml (25.0 mmol) of TEA. This mixture was refluxed at 80° C. for 2 hr. The solvents were removed and the residue dissolved in 100 ml of EtOAc, washed with brine and dried over MgSO₄. It was concentrated and purified by ISCO eluting with 0-5% MeOH/DCM to obtain 6.0 g (86%) of the desired product.

B. 1-Pyrimidin-2-yl-piperidin-4-yl-amine: To 5.9 g (21.2 mmol) of (1-pyrimidin-2-yl-piperidin-4-yl)-carboxylic acid tert-butyl ester dissolved in 100 ml of DCM at 0° C. was added 24.5 ml (318.3 mmol) of TFA. The ice bath was removed, and the mixture was stirred for 1 hr at rt. The solvents were removed, and the residue was dissolved in 200 ml of EtOAc and then washed with 60 ml of diluted NaHCO₃ solution. The organic layer was separated, and the aqueous layer was extracted three times with 150 ml portions of EtOAc. The combined organic layers were washed with brine, dried over MgSO₄ and filtered though a thin pad of silica gel. Solvents were removed to obtain 2.8 g (74%) of the desired product.

C. (4-Bromo-2-nitro-phenyl)-(1-pyrimidin-2-yl-piperidin-4-yl)-amine: To 1.0 g (5.61 mmol) of the 1-pyrimidin-2-yl-piperidin-4-yl-amine dissolved in 25 ml of DMF was added 1.48 g (6.74 mmol) of 3-bromo-2-fluoronitrobenzene and 1.55 g (11.2 mmol) of K₂CO₃. This mixture was heated to 120° C. for 2 hr. It was allowed to cool, and diluted with 80 ml of EtOAc, washed with water and brine. It was dried over MgSO₄ and concentrated. The crude mixture was purified by ISCO eluting with 0-5% MeOH/DCM to obtain 1.85 g (87%) of the strongly colored product.

D. (3′-Chloro-3-nitro-biphenyl-4-yl)-(1-pyrimidin-2-yl-piperidin-4-yl)-amine: To 420 mg (1.111 mmol) of (4-Bromo-2-nitro-phenyl)-(1-pyrimidin-2-yl-piperidin-4-yl)-amine dissolved in 12 ml of MeCN was added 208.4 mg (1.333 mmol) of 3-chloorophenylboronic acid, 306.6 mg (2.222 mmol) of K₂CO₃, 40 mg (0.056 mmol) of PdCl₂(PPh₃)₂ and 2 ml of water. This mixture was microwaved for 10 min at 140° C. It was diluted with 25 ml of EtOAc, washed with water and brine, and then dried over MgSO₄. It was concentrated and purified by preparative HPLC to obtain 406 mg (89%) of an orange powder. LC-MS [M+1]=410.0 (doublet); HPLC=2.73 min.

¹H NMR (400 MHz, chloroform-d) δ ppm 1.61-1.72 (m, 2H) 2.15-2.24 (dd, J=13.19, 3.61 Hz, 2H) 3.25-3.37 (ddd, J=13.77, 10.84, 2.93 Hz, 2H) 3.80-3.92 (m, 1H) 4.65 (dt, J=13.73, 3.59 Hz, 2H) 6.52 (t, J=4.69 Hz, 1H) 7.03 (d, J=9.18 Hz, 1H) 7.27-7.34 (m, 1H) 7.36 (t, J=7.82 Hz, 1H) 7.48 (td, J=1.76, 7.82 Hz 1H) 7.54 (t, J=1.76 Hz, 1H) 7.69 (dd, J=8.99, 2.15 Hz, 1H) 8.21 (d, J=7.03 Hz, 1H) 8.33 (d, J=4.88 Hz, 1H) 8.43 (d, J=2.15 Hz, 1H).

### 5.5. Preparation of 3′-Chloro-N⁴-(1-(pyrimidin-2-yl)piperidin-4-yl)biphenyl-3,4-diamine and N⁴-(1-(pyrimidin-2-yl)piperidin-4-yl)biphenyl-3,4-diamine

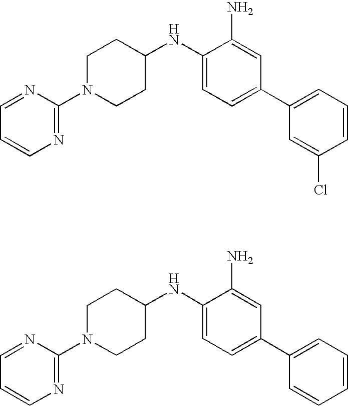

To 305 mg (0.74 mmol) of (3′-chloro-3-nitro-biphenyl-4-yl)-(1-pyrimidin-2-yl-piperidin-4-yl)-amine was added 78.4 mg (0.074 mmol) of 10% wt Pd on carbon, and 25 ml of EtOH. The air was completely removed, and the reaction mixture was vigorously stirred under hydrogen at rt for 2 hr. The reaction mixture was filtered and the concentrated. It was purified by preparatory HPLC to give 196 mg (69%) of 3′-chloro-N⁴-(1-(pyrimidin-2-yl)piperidin-4-yl)biphenyl-3,4-diamine [LC-MS [M+1]=380.0 (doublet); HPLC=2.02 min.] and 42 mg (16%) of N⁴-(1-pyrimidin-2-yl)piperidin-4-yl)biphenyl-3,4-diamine [LC-MS [M+1]=346.1; HPLC=1.85 min.]

### 5.6. Preparation of Additional Compounds

Additional compounds were prepared according to the general approach shown in Scheme 2:

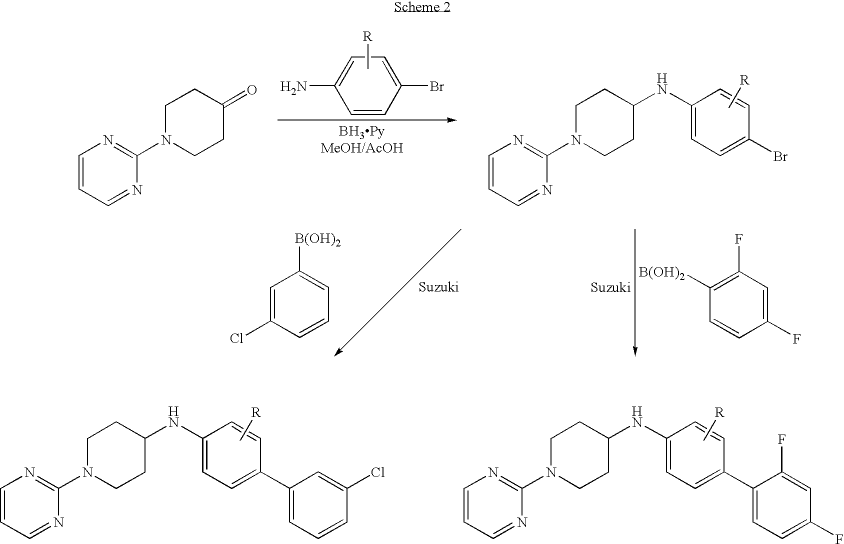

Descriptions of syntheses according to the first step shown in Scheme 2 are provided below:

A. (4-Bromo-2-fluoro-phenyl)-(1-pyrimidin-2-yl-piperidin-4-yl)-amine: To the mixture of bromoaniline (285 mg, 1.5 mmol) and ketone (266 mg, 1.5 mmol) in MeOH/AcOH (10:1, 5 ml) was added BH₃.Py in THF (8M, 188 μl). The mixture was stirred at rt for 2 h. The reaction mixture was concentrated and to the residue was added HCl (10%, 10 ml). The resulting mixture was stirred at rt for 30 min., then with cooling, the mixture was adjusted to alkaline with solid Na₂CO₃ and water. The aq. layer was extracted with EtOAc (5×30 ml). The EtOAc was dried (Na₂SO₄) and concentrated. The residue was subjected to ISCO to give the titled compound as a white solid (230 mg).

B. (4-Bromo-2-methyl-phenyl)-(1-pyrimidin-2-yl-piperidin-4-yl)-amine: To the mixture of bromoaniline (285 mg, 1.5 mmol) and ketone (266 mg, 1.5 mmol) in MeOH/AcOH (10:1, 5 ml) was added BH₃.Py in THF (8M, 188 μl). The mixture was stirred at rt for 2 h. The reaction mixture was concentrated and to the residue was added HCl (10%, 10 ml). The resulting mixture was stirred at rt for 30 min., then with cooling, the mixture was adjusted to alkaline with solid Na₂CO₃ and water. The aq. layer was extracted with EtOAc (5×30 ml). The EtOAc was dried (Na₂SO₄) and concentrated. The residue was subjected to ISCO to give the titled compound as a white solid (140 mg).

C. (4-Bromo-3-fluoro-phenyl)-(1-pyrimidin-2-yl-piperidin-4-yl)-amine: To the mixture of bromoaniline (1.5 mmol) and ketone (266 mg, 1.5 mmol) in MeOH/AcOH (10:1, 5 ml) was added BH₃.Py in THF (8M, 188 μl). The mixture was stirred at rt for 2 h. The reaction mixture was concentrated and to the residue was added HCl (10%, 10 ml). The resulting mixture was stirred at rt for 30 min., then with cooling, the mixture was adjusted to alkaline with solid Na₂CO₃ and water. The aq. layer was extracted with EtOAc (5×30 ml). The EtOAc was dried (Na₂SO₄) and concentrated. The residue was subjected to ISCO to give the titled compound as a white solid (150 mg).

D. (4-Bromo-3-methyl-phenyl)-(1-pyrimidin-2-yl-piperidin-4-yl)-amine: To the mixture of bromoaniline (1.5 mmol) and ketone (266 mg, 1.5 mmol) in MeOH/AcOH (10:1, 5 ml) was added BH₃.Py in THF (8M, 188 μl). The mixture was stirred at rt for 2 h. The reaction mixture was concentrated and to the residue was added HCl (10%, 10 ml). The resulting mixture was stirred at rt for 30 min., then with cooling, the mixture was adjusted to alkaline with solid Na₂CO₃ and water. The aq. layer was extracted with EtOAc (5×30 ml). The EtOAc was dried (Na₂SO₄) and concentrated. The residue was subjected to ISCO to give the titled compound as a white solid (150 mg).

The intermediates described below in Table 1 were prepared as follows: To 200 mg (1.130 mmol) of the appropriate ketone dissolved in 10 ml of 10% AcOH/MeOH was added 1.6 mmol (1.5 equiv) of the appropriate aniline, and then 0.14 ml (1.130 mmol) of 8 M BH₃.py solution. The reaction mixture was stirred at rt for 2 hr and then the solvents were evaporated. To the residue was added 5 ml of 1 N HCl and stirred vigorously for about 5 min, and then 20 ml of DCM was added. Solid Na₂CO₃ was used to adjust the pH to 9. The organic layer was separated, and the aqueous layer was extracted twice with DCM. The combined organic layers were washed with brine and dried over MgSO₄. It was concentrated and purified by ISCO eluting with 5-35% EtOAc/hex. Yields on these reactions were all over 80%, except with the cyano anilines where the yields were about 22%.

TABLE 1
(4-Bromo-2-cyano-phenyl)-(1-pyrimidin-2-yl-piperidin-4-yl)-amine
(4-Bromo-2-chloro-phenyl)-(1-pyrimidin-2-yl-piperidin-4-yl)-amine
(4-Bromo-2-trifluoromethyl-phenyl)-(1-pyrimidin-2-yl-piperidin-4-yl)-
amine
(4-Bromo-3-chloro-2-methyl-phenyl)-(1-pyrimidin-2-yl-piperidin-4-yl)-
amine
(4-Bromo-3-chloro-phenyl)-(1-pyrimidin-2-yl-piperidin-4-yl)-amine
(4-Bromo-3-trifluoromethyl-phenyl)-(1-pyrimidin-2-yl-piperidin-4-yl)-
amine
(4-Bromo-3-methoxy-phenyl)-(1-pyrimidin-2-yl-piperidin-4-yl)-amine

The products listed below in Table 2 were prepared by Suzuki coupling, as follows: A mixture of 1.0 equivalent of the aryl bromide, 1.2 equivalents of the boronic acid, 2.0 equivalents of K₂CO₃, 0.05 equivalents of PdCl₂(PPh₃)₂, and small volume of 20% water/MeCN (15 ml per mmol of aryl bromide) was microwaved at 140° C. for 10 minutes. The reaction mixture was diluted with EtOAc, and washed with water and brine. It was dried over MgSO₄, concentrated and then purified by preparative HPLC to obtain the desired products, with yields between 58-92%.

TABLE 2

| | LC-MS | HPLC |
|---|---|---|
| Compound | [M + 1] | [min.] |
| (3′-Chloro-3-cyano-biphenyl-4-yl)-(1-pyrimidin-2-yl- | 390.0 | 2.51 |
| piperidin-4-yl)-amine | | |
| (3′-Chloro-2-methoxy-biphenyl-4-yl)-(1-pyrimidin-2- | 395.1 | 2.24 |
| yl-piperidin-4-yl)-amine | | |
| (3′-Chloro-3-chloro-biphenyl-4-yl)-(1-pyrimidin-2-yl- | 399.0 | 2.83 |
| piperidin-4-yl)-amine | | |
| (3′-Chloro-2-chloro-biphenyl-4-yl)-(1-pyrimidin-2-yl- | 399.1 | 3.45 |
| piperidin-4-yl)-amine | | |
| (2,3′-Dichloro-3-methyl-biphenyl-4-yl)-(1-pyrimidin- | 413.1 | 2.69 |
| 2-yl-piperidin-4-yl)-amine | | |
| (3′-Chloro-3-trifluoromethyl-biphenyl-4-yl)-(1- | 433.1 | 2.83 |
| pyrimidin-2-yl-piperidin-4-yl)-amine | | |
| (3′-Chloro-2-trifluoromethyl-biphenyl-4-yl)-(1- | 433.0 | 2.54 |
| pyrimidin-2-yl-piperidin-4-yl)-amine | | |
| (3′-Chloro-3-methyl-biphenyl-4-yl)-(1-pyrimidin-2-yl- | 379.0 | 2.48 |
| piperidin-4-yl)-amine | | |
| (3′-Chloro-2-methyl-biphenyl-4-yl)-(1-pyrimidin-2-yl- | 379.1 | 2.15 |
| piperidin-4-yl)-amine | | |
| (3′-Chloro-3-fluoro-biphenyl-4-yl)-(1-pyrimidin-2-yl- | 383.0 | 2.52 |
| piperidin-4-yl)-amine | | |
| (3′-Chloro-2-fluoro-biphenyl-4-yl)-(1-pyrimidin-2-yl- | 383.1 | 2.50 |
| piperidin-4-yl)-amine | | |
| (3-Cyano-2′,4′-difluoro-biphenyl-4-yl)-(1-pyrimidin- | 392.1 | 2.39 |
| 2-yl-piperidin-4-yl)-amine | | |
| (2′,4′-Difluoro-2-methoxy-biphenyl-4-yl)-(1- | 397.1 | 3.20 |
| pyrimidin-2-yl-piperidin-4-yl)-amine | | |
| (3-Chloro-2′4′-difluoro-biphenyl-4-yl)-(1-pyrimidin-2- | 401.0 | 2.61 |
| yl-piperidin-4-yl)-amine | | |
| (2-Chloro-2′,4′-difluoro-biphenyl-4-yl)-(1-pyrimidin- | 401.1 | 2.43 |
| 2-yl-piperidin-4-yl)-amine | | |
| (2-Chloro-2′,4′-difluoro-3-methyl-biphenyl-4-yl)-(1- | 415.0 | 2.53 |
| pyrimidin-2-yl-piperidin-4-yl)-amine | | |
| (2′,4′-Difluoro-3-trifluoromethyl-biphenyl-4-yl)-(1- | 435.5 | 2.64 |
| pyrimidin-2-yl-piperidin-4-yl)-amine | | |
| (2′,4′-Difluoro-2-trifluoromethyl-biphenyl-4-yl)-(1- | 435.0 | 2.42 |
| pyrimidin-2-yl-piperidin-4-yl)-amine | | |
| (2′,4′-Difluoro-3-methyl-biphenyl-4-yl)-(1-pyrimidin- | 381.1 | 2.33 |
| 2-yl-piperidin-4-yl)-amine | | |
| (2′,4′-Difluoro-2-methyl-biphenyl-4-yl)-(1-pyrimidin- | 381.0 | 2.05 |
| 2-yl-piperidin-4-yl)-amine | | |
| (1-Pyrimidin-2-yl-piperidin-4-yl)-(3,2′,4′-trifluoro- | 385.0 | 2.47 |
| biphenyl-4-yl)-amine | | |
| (1-Pyrimidin-2-yl-piperidin-4-yl)-(2,2′,4′-trifluoro- | 385.0 | 2.36 |
| biphenyl-4-yl)-amine | | |
| (5-Chloro-2′-fluoro-3-methyl-biphenyl-4-yl)-(1- | 397.0 | 2.37 |
| pyrimidin-2-yl-piperidin-4-yl)-amine | | |
| (5′-Chloro-3,2′-difluoro-biphenyl-4-yl)-(1-pyrimidin- | 401.0 | 2.51 |
| 2-yl-piperidin-4-yl)-amine | | |
| (5′-Chloro-2,2′-difluoro-biphenyl-4-yl)-(1-pyrimidin- | 401.0 | 2.41 |
| 2-yl-piperidin-4-yl)-amine | | |
| (4′-Chloro-3-methyl-biphenyl-4-yl)-(1-pyrimidin-2-yl- | 379.1 | 2.26 |
| piperidin-4-yl)-amine | | |
| (4′-Chloro-2-methyl-biphenyl-4-yl)-(1-pyrimidin-2-yl- | 379.0 | 2.11 |
| piperidin-4-yl)-amine | | |
| (4′-Chloro-3-fluoro-biphenyl-4-yl)-(1-pyrimidin-2-yl- | 383.0 | 2.51 |
| piperidin-4-yl)-amine | | |
| (4′-Chloro-2-fluoro-biphenyl-4-yl)-(1-pyrimidin-2-yl- | 383.0 | 2.46 |
| piperidin-4-yl)-amine | | |

LC-MS data was obtained under the following conditions: Waters ZQ LC/MS, Column: Sunfire C18 5μ 5 cm×4.6 mm ID, Solvent A: acetonitrile; Solvent B: 10 mM ammonium acetate in water. HPLC data was obtained using the following conditions: Discovery Analytical System; Shim-pack VP ODS 4.6×50 mm; Solvent A: Water+0.1% TFA; Solvent B: MeOH+0.1% TFA; start % B=10, final % B=90; wavelength: 220; gradient time: 2 min; flow rate: 3.5 ml/min.

### 5.7. Human Proline Transporter Assay

The ability of compounds to inhibit the proline transporter was determined as follows. A human SLC6A7 cDNA was cloned into a pcDNA3.1 vector and transfected into COS-1 cells. A cell clone stably expressing proline transporter was selected for the assay.

Transfected cells were seeded at 15,000 cells per well in a 384 well plate and grown overnight. The cells were then washed with Krebs-Ringer's-HEPES-Tris (KRHT) buffer, pH 7.4, containing 120 mM NaCl, 4.7 mM KCl, 2.2 mM CaCl, 1.2 mM MgSO₄, 1.2 mM KH₂PO₄, 10 mM HEPES and 5 mM Tris. The cells were then incubated with 50 μl of KRHT buffer containing 45 nM ³H-Proline for 20 minutes at room temperature. Radiolabeled proline uptake was terminated by removing the radiolabeled proline and washing the cells rapidly three times with 100 μl of ice-cold KRHT buffer. Scintillation fluid (50 μl) was added per well, and the amount of tritiated proline present was determined using a Packard TopCount Scintillation counter.

Nonspecific uptake was determined by measuring of ³H-proline uptake in the presence of 2 mM cold proline.

The IC₅₀ of a compound was determined by measuring inhibition of four separate samples at ten concentrations, typically beginning with 10 μM followed by nine three-fold dilutions (i.e., 10, 3.3, 1.1, 0.37, 0.12, 0.41, 0.014, 0.0046, 0.0015, and 0 μM). Percent inhibitions were calculated against the control. The IC₅₀ of a compound was determined using the ten data points, each of which was an average of the four corresponding measurements.

### 5.8. Murine Proline Transporter Assay

Forebrain tissue was dissected from a wild type mouse and homogenized in 7 ml ice-cold homogenization buffer: 0.32 M sucrose, 1 mM NaHCO₃, protease inhibitor cocktail (Roche).

The brain homogenates were centrifuged at 1000×g for 10 min to remove nuclei. Supernatant was collected and re-centrifuged at 20000×g for 20 min to pellet crude synaptosomes. The synaptosomes were resuspended in ice-cold assay buffer: 122 mM NaCl, 3.1 mM KCl, 25 mM HEPES, 0.4 mM KH₂PO₄, 1.2 mM MgSO₄, 1.3 mM CaCl₂, 10 mM dextrose at pH 7.4. Resuspended synaptosomes were centrifuged again at 20000×g for 20 minutes, and pelleted synaptosomes were resuspended in assay buffer. Protein concentration was measured by DC protein assay kit (BioRad).

Proline transport assay was performed in 100 μl reaction mix consisting of 10 μg synaptosomes, 1 μCi/0.24 μM [H3]-proline in assay buffer for a time between 0 to 20 minutes at room temperature. The reaction was terminated by rapid filtration through GF/B filter plate (Millipore) followed by three rapid washes in 200 ul ice-cold assay buffer. Fifty microliters of Microscint-20 was added to each reaction and incubated for 2 hours. The [H3]-proline transport was determined by radioactivity counting.

To determine proline transport inhibition by compounds, compounds were incubated with the reaction mixture at concentrations ranging from 0 to 10 μM (11 points, beginning at 10 um; 3-fold dilutions; 4 replicates averaged to provide one point). The baseline activity, or nonspecific activity, was measured in the presence of 0.3 mM GGFL (Enkephalin, Sigma) in the reaction. The nonspecific activity was also measured in synaptosomes of SLC6A7 knockout mice. The nonspecific activities measured by the two methods were found to be identical.

### 5.9. Human Dopamine Transporter Assay

The ability of compounds to inhibit the dopamine transporter was determined as follows. A human DAT cDNA (NM_{—}001044) was cloned into a pcDNA3.1 vector and transfected into COS-1 cells. The resulting cell lines that stably express the dopamine transporter were used for further experimentation.

Transfected cells were seeded at 15,000 cells per well in a 384 well plate and grown overnight. The cells were then washed with Krebs-Ringer's-HEPES-Tris (KRHT) buffer, pH 7.4, containing 125 mM NaCl, 4.8 mM KCl, 1.3 mM CaCl₂, 1.2 mM MgSO₄ 10 mM D-glucose, 25 mM HEPES, 1 mM sodium ascorbate and 1.2 mM KH₂PO₄. The cells were then incubated with 50 μl of KRHT buffer containing 1 μM ³H-Dopamine for 10 minutes at room temperature. Radiolabeled dopamine uptake was terminated by removing the radiolabeled dopamine and washing the cells rapidly three times with 100 μl of ice-cold KRHT buffer. Scintillation fluid (50 μl) was added per well and the amount of tritiated dopamine present was determined using a Packard TopCount Scintillation counter.

Nonspecific uptake was determined by measuring of ³H-dopamine uptake in the presence of 250 μM benztropine. The IC₅₀ of a compound was determined by measuring inhibition of four separate samples at ten concentrations, typically beginning with 10 μM followed by nine three-fold dilutions (i.e., 10, 3.3, 1.1, 0.37, 0.12, 0.41, 0.014, 0.0046, 0.0015, and 0 μM). Percent inhibitions were calculated against the control. The percentage inhibitions were calculated against the control, and the average of the quadruplicates was used for IC₅₀ calculation.

### 5.10. Human Glycine Transporter Assay

The ability of compounds to inhibit the glycine transporter was determined as follows. A human glycine transporter cDNA (NM_{—}006934) was cloned into a pcDNA3.1 vector and transfected into COS-1 cells. The resulting cell lines that stably express the glycine transporter were used for further experimentation.

Transfected cells were seeded at 15,000 cells per well in a 384 well plate and grown overnight. The cells were then washed with Krebs-Ringer's-HEPES-Tris (KRHT) buffer, pH 7.4, containing 120 mM NaCl, 4.7 mM KCl, 2.2 mM CaCl₂, 1.2 mM MgSO₄, 1.2 mM KH₂PO₄, 10 mM HEPES and 5 mM Tris. The cells were then incubated with 50 μl of KRHT buffer containing 166 nM ³H-glycine for 10 minutes at room temperature. Radiolabeled glycine uptake was terminated by removing the radiolabeled glycine and washing the cells rapidly three times with 100 μl of ice-cold KRHT buffer. Scintillation fluid (50 μl) was added per well and the amount of tritiated glycine present was determined using a Packard TopCount Scintillation counter.

Nonspecific uptake was determined by measuring ³H-glycine uptake in the presence of 2 mM cold glycine. The IC₅₀ of a compound was determined by measuring inhibition of four separate samples at ten concentrations, typically beginning with 10 μM followed by nine three-fold dilutions (i.e., 10, 3.3, 1.1, 0.37, 0.12, 0.41, 0.014, 0.0046, 0.0015, and 0 μM). Percent inhibitions were calculated against the control. The percentage inhibitions were calculated against the control, and the average of the quadruplicates was used for IC₅₀ calculation.

### 5.11. Calculating IC₅₀ Values

The IC₅₀ of a compound with regard to a given target is determined by fitting the relevant data, using the Levenburg Marquardt algorithm, to the equation:

y=A+((B−A)/(1+((C/x)̂D)))

wherein A is the minimum y value; B is the maximum y value; C is the IC₅₀; and D is the slope. The calculation of the IC₅₀ is performed using XLFit4 software (ID Business Solutions Inc., Bridgewater, N.J. 08807) for Microsoft Excel (the above equation is model 205 of that software).

Each of the references (e.g., patents and patent applications) cited herein is incorporated herein in its entirety.

What is claimed is:

1. A compound of formula I: 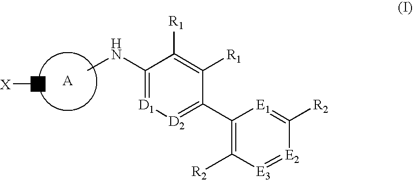
or a pharmaceutically acceptable salt or solvate thereof, wherein:
A is an optionally substituted non-aromatic heterocycle;
each of D₁ and D₂ is independently N or CR₁;
each of E₁, E₂ and E₃ is independently N or CR₂;
X is optionally substituted heteroaryl;
each R₁ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A};
each R₂ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A};
each R_{A} is independently hydrogen or optionally substituted alkyl, aryl, arylalkyl, alkylaryl, heterocycle, heterocycle-alkyl, or alkyl-heterocycle; and
each R_{B} is independently hydrogen or optionally substituted alkyl, aryl, arylalkyl, alkylaryl, heterocycle, heterocycle-alkyl, or alkyl-heterocycle.

2. The compound of claim 1, which is a potent proline transporter inhibitor.

3. The compound of claim 2, which has a PTIC₅₀ of less than about 150 nM.

4. The compound of claim 3, which has a PTIC₅₀ of less than about 100 nM.

5. The compound of claim 4, which has a PTIC₅₀ of less than about 50 nM.

6. The compound of claim 1, which has a DTIC₅₀ of greater than about 1 μM.

7. The compound of claim 1, which has a GTIC₅₀ of greater than about 1 μM.

8. The compound of claim 1, wherein A is monocyclic.

9. The compound of claim 1, wherein A is bicyclic.

10. The compound of claim 1, wherein A is unsubstituted.

11. The compound of claim 1, wherein A is optionally substituted pyrrolidine, piperidine, hexahydropyrimidine, 1,2,3,6-tetrahydropyridine, octahydrocyclopenta[c]pyrrole, or octahydropyrrolo[3,4-c]pyrrole.

12. The compound of claim 1, wherein one of D₁ and D₂ is N.

13. The compound of claim 1, wherein both D₁ and D₂ are N.

14. The compound of claim 1, wherein both D₁ and D₂ are CR₁.

15. The compound of claim 1, wherein one of E₁, E₂ and E₃ is N.

16. The compound of claim 1, wherein two of E₁, E₂ and E₃ are N.

17. The compound of claim 1, wherein all of E₁, E₂ and E₃ are N.

18. The compound of claim 1, wherein all of E₁, E₂ and E₃ are independently CR₂.

19. The compound of claim 1, wherein R₁ is hydrogen, halogen, or optionally substituted alkyl.

20. The compound of claim 1, wherein R₁ is OR_{A}.

21. The compound of claim 20, wherein R_{A} is hydrogen or optionally substituted alkyl.

22. The compound of claim 1, wherein R₂ is hydrogen, halogen, or optionally substituted alkyl.

23. The compound of claim 1, wherein R₂ is OR_{A}.

24. The compound of claim 23, wherein R_{A} is hydrogen or optionally substituted alkyl.

25. The compound of claim 1, wherein X is an optionally substituted 5-, 6-, 9- or 10-membered heteroaryl.

26. The compound of claim 25, wherein X is optionally substituted 5- or 6-membered heteroaryl.

27. The compound of claim 26, wherein X is of the formula: 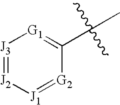
wherein:
each of G₁ and G₂ are independently N or CR₃;
each of J₁, J₂ and J₃ are independently N or CR₄;
each R₃ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A}; and
each R₄ is independently hydrogen, halogen, cyano, R_{A}, OR_{A}, C(O)R_{A}, C(O)OR_{A}, C(O)N(R_{A}R_{B}), N(R_{A}R_{B}), or SO₂R_{A};
provided that at least one of J₁, J₂ and J₃ is CR₄.

28. The compound of claim 27, wherein one of G₁ and G₂ is N.

29. The compound of claim 27, wherein both G₁ and G₂ are N.

30. The compound of claim 27, wherein both G₁ and G₂ are CR₃.

31. The compound of claim 27, wherein one of J₁, J₂ and J₃ is N.

32. The compound of claim 27, wherein two of J₁, J₂ and J₃ are N.

33. The compound of claim 27, wherein all of J₁, J₂ and J₃ are independently CR₄.

34. The compound of claim 27, wherein R₃ is hydrogen, halogen, or optionally substituted alkyl.

35. The compound of claim 27, wherein R₃ is OR_{A}.

36. The compound of claim 35, wherein R_{A} is hydrogen or optionally substituted alkyl.

37. The compound of claim 27, wherein R₄ is hydrogen, halogen, or optionally substituted alkyl.

38. The compound of claim 27, wherein R₄ is OR_{A}.

39. The compound of claim 38, wherein R_{A} is hydrogen or optionally substituted alkyl.

40. The compound of claim 27, which is of formula I(A):

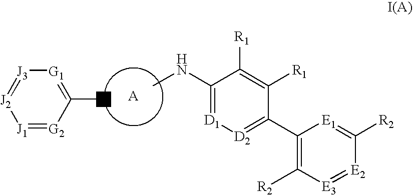

41. The compound of claim 40, which is of formula I(B): 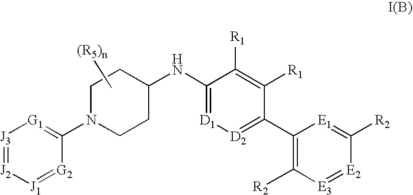
wherein:
each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A};
each R_{5A} is independently hydrogen or optionally substituted alkyl, aryl, arylalkyl, alkylaryl, heterocycle, heterocycle-alkyl, or alkyl-heterocycle;
each R_{5B} is independently hydrogen or optionally substituted alkyl, aryl, arylalkyl, alkylaryl, heterocycle, heterocycle-alkyl, or alkyl-heterocycle; and
n is 0-5.

42. The compound of claim 40, which is of formula I(C): 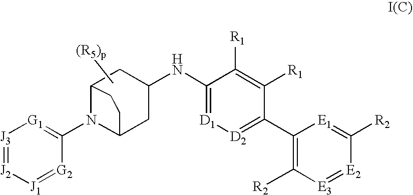
wherein:
each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A};
each R_{5A} is independently hydrogen or optionally substituted alkyl, aryl, arylalkyl, alkylaryl, heterocycle, heterocycle-alkyl, or alkyl-heterocycle;
each R_{5B} is independently hydrogen or optionally substituted alkyl, aryl, arylalkyl, alkylaryl, heterocycle, heterocycle-alkyl, or alkyl-heterocycle; and
p is 0-7.

43. The compound of claim 40, which is of formula I(D): 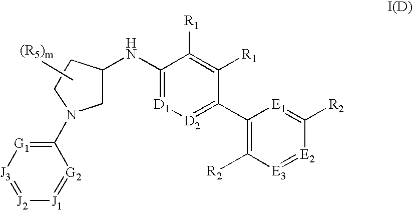
wherein:
each R₅ is independently halogen, cyano, R_{5A}, OR_{5A}, C(O)R_{5A}, C(O)OR_{5A}, C(O)N(R_{5A}R_{5B}), N(R_{5A}R_{5B}), or SO₂R_{5A};
each R_{5A} is independently hydrogen or optionally substituted alkyl, aryl, arylalkyl, alkylaryl, heterocycle, heterocycle-alkyl, or alkyl-heterocycle;
each R_{5B} is independently hydrogen or optionally substituted alkyl, aryl, arylalkyl, alkylaryl, heterocycle, heterocycle-alkyl, or alkyl-heterocycle; and
m is 0-4.

44. A compound or a pharmaceutically acceptable salt thereof, wherein the compound is:
N-(3′-chloro-3-methylbiphenyl-4-yl)-1-(pyrimidin-2-yl)piperidin-4-amine;
N-(4′-chloro-2′-fluorobiphenyl-4-yl)-8-(pyrimidin-2-yl)-8-azabicyclo[3.2.1]octan-3-amine;
N-(2′,4′-difluorobiphenyl-4-yl)-1-(pyrimidin-2-yl)piperidin-4-amine;
(3′-chloro-3-nitro-biphenyl-4-yl)-(1-pyrimidin-2-yl-piperidin-4-yl)-amine;
3′-chloro-N⁴-(1-(pyrimidin-2-yl)piperidin-4-yl)biphenyl-3,4-diamine;
N⁴-(1-(pyrimidin-2-yl)piperidin-4-yl)biphenyl-3,4-diamine;
(3′-chloro-3-cyano-biphenyl-4-yl)-(1-pyrimidin-2-yl-piperidin-4-yl)-amine;
(3′-chloro-2-methoxy-biphenyl-4-yl)-(1-pyrimidin-2-yl-piperidin-4-yl)-amine;
(3′-chloro-3-chloro-biphenyl-4-yl)-(1-pyrimidin-2-yl-piperidin-4-yl)-amine;
(3′-chloro-2-chloro-biphenyl-4-yl)-(1-pyrimidin-2-yl-piperidin-4-yl)-amine;
(2,3′-dichloro-3-methyl-biphenyl-4-yl)-(1-pyrimidin-2-yl-piperidin-4-yl)-amine;
(3′-chloro-3-trifluoromethyl-biphenyl-4-yl)-(1-pyrimidin-2-yl-piperidin-4-yl)-amine;
(3′-chloro-2-trifluoromethyl-biphenyl-4-yl)-(1-pyrimidin-2-yl-piperidin-4-yl)-amine;
(3′-chloro-3-methyl-biphenyl-4-yl)-(1-pyrimidin-2-yl-piperidin-4-yl)-amine;
(3′-chloro-2-methyl-biphenyl-4-yl)-(1-pyrimidin-2-yl-piperidin-4-yl)-amine;
(3′-chloro-3-fluoro-biphenyl-4-yl)-(1-pyrimidin-2-yl-piperidin-4-yl)-amine;
(3′-chloro-2-fluoro-biphenyl-4-yl)-(1-pyrimidin-2-yl-piperidin-4-yl)-amine;
(3-cyano-2′,4′-difluoro-biphenyl-4-yl)-(1-pyrimidin-2-yl-piperidin-4-yl)-amine;
(2′,4′-difluoro-2-methoxy-biphenyl-4-yl)-(1-pyrimidin-2-yl-piperidin-4-yl)-amine;
(3-chloro-2′4′-difluoro-biphenyl-4-yl)-(1-pyrimidin-2-yl-piperidin-4-yl)-amine;
(2-chloro-2′,4′-difluoro-biphenyl-4-yl)-(1-pyrimidin-2-yl-piperidin-4-yl)-amine;
(2-chloro-2′,4′-difluoro-3-methyl-biphenyl-4-yl)-(1-pyrimidin-2-yl-piperidin-4-yl)-amine;
(2′,4′-difluoro-3-trifluoromethyl-biphenyl-4-yl)-(1-pyrimidin-2-yl-piperidin-4-yl)-amine;
(2′,4′-difluoro-2-trifluoromethyl-biphenyl-4-yl)-(1-pyrimidin-2-yl-piperidin-4-yl)-amine;
(2′,4′-difluoro-3-methyl-biphenyl-4-yl)-(1-pyrimidin-2-yl-piperidin-4-yl)-amine;
(2′,4′-difluoro-2-methyl-biphenyl-4-yl)-(1-pyrimidin-2-yl-piperidin-4-yl)-amine;
(1-pyrimidin-2-yl-piperidin-4-yl)-(3,2′,4′-trifluoro-biphenyl-4-yl)-amine;
(1-pyrimidin-2-yl-piperidin-4-yl)-(2,2′,4′-trifluoro-biphenyl-4-yl)-amine;
(5-chloro-2′-fluoro-3-methyl-biphenyl-4-yl)-(1-pyrimidin-2-yl-piperidin-4-yl)-amine;
(5′-chloro-3,2′-difluoro-biphenyl-4-yl)-(1-pyrimidin-2-yl-piperidin-4-yl)-amine;
(5′-chloro-2,2′-difluoro-biphenyl-4-yl)-(1-pyrimidin-2-yl-piperidin-4-yl)-amine;
(4′-chloro-3-methyl-biphenyl-4-yl)-(1-pyrimidin-2-yl-piperidin-4-yl)-amine;
(4′-chloro-2-methyl-biphenyl-4-yl)-(1-pyrimidin-2-yl-piperidin-4-yl)-amine;
(4′-chloro-3-fluoro-biphenyl-4-yl)-(1-pyrimidin-2-yl-piperidin-4-yl)-amine; or
(4′-chloro-2-fluoro-biphenyl-4-yl)-(1-pyrimidin-2-yl-piperidin-4-yl)-amine.

45. A pharmaceutical composition comprising a compound of claim 1 and a pharmaceutically acceptable excipient.

46. A single unit dosage form comprising the pharmaceutical composition of claim 45.

47. A method of inhibiting a proline transporter, which comprises contacting a proline transporter with sufficient amount of a compound of claim 1.

48. The method of claim 47, wherein the proline transporter is encoded by the human gene SLC6A7.

49. A method of improving the cognitive performance of a human patient, which comprises administering to the patient an amount of a compound of claim 1 sufficient to improve the cognitive performance.

50. The method of claim 49, wherein the cognitive performance is rapidity of learning, comprehension, reasoning, or memory.

51. A method of treating, managing or preventing a cognitive disorder, memory loss, or a learning disorder in a human patient, which comprises administering to the patient a therapeutically or prophylactically effective amount of a compound of claim 1.

52. A method of treating, managing or preventing a disease or disorder in a patient, which comprises administering to the patient a therapeutically or prophylactically effective amount of a compound of claim 1, wherein the disease or disorder is age-associated memory impairment, Alzheimer's disease, Attention-Deficit/Hyperactivity Disorder, autism, Down syndrome, Fragile X syndrome, Huntington's disease, Parkinson's disease, or schizophrenia.

53. A method of treating, managing or preventing dementia in a patient, which comprises administering to the patient a therapeutically or prophylactically effective amount of a compound of claim 1.

54. The method of claim 53, wherein the dementia is associated with a metabolic-toxic, structural or infectious cause.
